(19) United States
(12) Patent Application Publication
(10) Pub. No.: US 2026/0207330 A1
(43) Pub. Date: Jul. 23, 2026

(54) PROSTHETIC VALVE SUPPORT WITH FLOW CHANNELS

(71) Applicant: Edwards Lifesciences Corporation, Irvine, CA (US)

(72) Inventor: Michael G. Valdez, Riverside, CA (US)

(21) Appl. No.: 19/570,493

(22) Filed: Mar. 18, 2026

Related U.S. Application Data
(63) Continuation of application No. PCT/US2024/047454, filed on Sep. 19, 2024.
(60) Provisional application No. 63/584,306, filed on Sep. 21, 2023.

(51) Int. Cl. A61F 2/24

(52) U.S. Cl. CPC A61F 2/2418; A61F 2/2436; A61F 2230/0006; A61F 2230/0008; A61F 2230/0069; A61F 2250/0029; A61F 2250/0069; A61F 2250/0098

(57) ABSTRACT

An implant may include a framework of struts forming a tunnel, where the framework of struts includes an elbow separating a first span and a second span of the implant; a first opening to the tunnel associated with the first span including a generally circular-shaped opening; and a second opening to the tunnel associated with the second span including a generally oval-shaped opening.

### RELATED APPLICATIONS

This application is a continuation of PCT Patent Application No. PCT/2024/047454 filed on Sep. 19, 2024, which claims the benefit of and priority to U.S. Provisional Patent Application Ser. No. 63/584,306 filed on Sep. 21, 2023, each of these applications being incorporated by reference in its entirety herein.

### FIELD

The present disclosure relates to one or more devices that provide prosthetic valve support with flow channels.

### BACKGROUND

Prosthetic heart valves may be used to treat cardiac valvular disorders. The native heart valves (the aortic, pulmonary, tricuspid, and mitral valves) function to prevent backward flow or regurgitation, while allowing forward flow. These heart valves may be rendered less effective by congenital, inflammatory, infectious conditions, etc. Such conditions may eventually lead to serious cardiovascular compromise or death. For many years, doctors attempted to treat such disorders with surgical repair or replacement of the valve during open heart surgery.

A transcatheter technique for introducing and implanting a prosthetic heart valve using a catheter in a manner that is less invasive than open heart surgery may reduce complications associated with open heart surgery. In this technique, a prosthetic valve may be mounted in a crimped state on the end portion of a catheter and advanced through a blood vessel of the patient until the valve reaches the implantation site. The valve at the catheter tip may then be expanded to its functional size at the site of the defective native valve, such as by inflating a balloon on which the valve is mounted or, for example, the valve may have a resilient, self-expanding stent or frame that expands the valve to its functional size when it is advanced from a delivery sheath at the distal end of the catheter. Optionally, the valve may have a balloon-expandable frame, self-expanding frame, a mechanically-expandable frame, and/or a frame expandable in multiple or a combination of ways.

Transcatheter heart valves (THVs) may be appropriately sized for placement inside many native cardiac valves or orifices. However, with larger native valves, blood vessels (e.g., an enlarged aorta), grafts, etc., aortic transcatheter valves might be too small to secure into the larger implantation or deployment site. In this case, the transcatheter valve may not be large enough to sufficiently expand inside the native valve or other implantation or deployment site or the implantation/deployment site may not provide a good seat for the THV to be secured in place. As one example, aortic insufficiency may be associated with difficulty securely implanting a THV in the aorta and/or aortic valve. Accordingly, there exists a need for improved systems and methods of securing a THV in a relatively large diameter blood vessel or annulus.

### SUMMARY

Certain embodiments of the disclosure pertain to devices related to docking stations, frame adaptors, prestents, and the like for engaging and retaining a prosthetic implant such as a prosthetic heart valve in a lumen of the body, such as a blood vessel or valve of the heart. For example, some embodiments may relate to an implant that is configured to provide a flow channel from a vessel that might otherwise be occluded by the docking station, frame adaptor, and/or prestent.

One or more embodiments of the present disclosure may include an implant that may include a framework of struts forming a tunnel, where the framework of struts includes an elbow separating a first span and a second span of the implant. The implant may also include a first opening to the tunnel associated with the first span that includes a generally circular-shaped opening. The implant may also include a second opening to the tunnel associated with the second span that may include a generally oval-shaped opening.

One or more embodiments of the present disclosure may include a method of deploying an artificial valve. The method may include guiding a delivery catheter through a body of a patient to a first hepatic vein. The method may also include deploying a first implant so that a first span of the first implant is disposed within the first hepatic vein and a second span of the first implant is disposed outside of the first hepatic vein, where a first elbow separates the first span and the second span of the first implant. The method may also include deploying a valve framework at a junction between an inferior vena cava (IVC) and a right atrium (RA) of the patient, the valve framework abutting at least part of the second span of the first implant.

The foregoing and other objects, features, and advantages of the described technology will become more apparent from the following detailed description, which proceeds with reference to the accompanying figures.

### BRIEF DESCRIPTION OF THE DRAWINGS

FIG. 1A is a top view of an implant that provides prosthetic valve support with a flow channel.

FIG. 1B is a bottom view of the implant of FIG. 1A.

FIG. 1C is a side view of the implant of FIG. 1A.

FIG. 1D is a front view of the implant of FIG. 1A.

FIG. 1E is a back view of the implant of FIG. 1A.

FIG. 2A is a cutaway view of a patient proximate the inferior vena cava (IVC) and the right atrium (RA) with a deployed docking station and valve, and with the implant of FIG. 1A-1E also deployed.

FIG. 2B is a view of the patient of FIG. 2A along the view line A-A illustrated in FIG. 2A.

FIG. 3A illustrates a side view of the implant of FIGS. 1A-1E loaded on a delivery catheter.

FIG. 3B is a view of the implant loaded on the delivery catheter as shown in FIG. 3A along the view line B-B illustrated in FIG. 3A.

FIG. 4 is a side view of the implant of FIG. 1A-1E with a close-up view of struts with different thicknesses in the same device.

FIGS. 5A and 5B illustrate a side view of an implant with different openings to create the flow channel.

FIGS. 6A and 6B illustrate an example implant that includes radiopaque markers to facilitate placement and/or orientation of the example implant.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

It should be understood that the disclosed embodiments may be adapted for support of devices delivered and/or implanted in any of the native annuluses and blood vessels of the heart (e.g., the pulmonary, mitral, and tricuspid annuluses, the inferior and superior vena cava, etc.), and may be used with any of various delivery approaches (e.g., retrograde, antegrade, transseptal, transventricular, transatrial, etc.).

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods may be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, may be used in combination with any other feature described herein.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

In the context of the present application, the terms “lower” and “upper” are used interchangeably with the terms “inflow” and “outflow”, respectively. Thus, for example, typically the lower end of a valve or docking station as depicted in the figures is its inflow end and the upper end of the valve or docking station is its outflow end unless explicitly described otherwise.

As used herein, the term “proximal” refers to a position, direction, or portion of a device that is closer to the user and further away from the implantation site and/or body lumen orifice. As used herein, the term “distal” refers to a position, direction, or portion of a device that is further away from the user and closer to the implantation site and/or body lumen orifice. Thus, for example, proximal motion of a device is motion of the device toward the user, while distal motion of the device is motion of the device away from the user.

The terms “longitudinal” and “axial” refer to an axis extending in the upstream and downstream directions, or in the proximal and distal directions, unless otherwise expressly defined.

Although there are alternatives for various components, features, parameters, operating conditions, etc., set forth herein, that does not mean that those alternatives are necessarily equivalent and/or perform equally well. Nor does it mean that the alternatives are listed in a preferred order unless stated otherwise.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part may become a “lower” part simply by turning the object over. Nevertheless, it is still the same part and the object remains the same. As used herein, “and/or” means “and” or “or”, as well as “and” and “or”.

As used herein, the terms “integrally formed” and “unitary construction” refer to a construction that does not require any sutures, fasteners, or other securing means to attach two portions of the construction together.

### Examples of Disclosed Technology

The present disclosure pertains to devices to support valve adapter/docking station/landing zone/prestent technology for implanting a prosthetic heart valve, such as a transcatheter heart valve, in a lumen or valve of the heart where the diameter of the lumen or valve is significantly greater than the functional diameter of the prosthetic valve. In certain examples, the docking station may comprise a radially expandable and collapsible frame formed from a plurality of struts, and including a valve seat within the frame configured to receive an expandable prosthetic valve. In certain embodiments, the valve seat may comprise a plurality of struts coupled to the frame and angled inwardly toward the longitudinal axis of the frame. The valve seat may be configured to engage and retain prosthetic valves of a variety of types and sizes. The outer aspect of the docking station frame may engage the surrounding tissue of the native lumen and form a seal, and the valve seat may engage and retain the prosthetic heart valve within the docking station. In certain embodiments, the frame may comprise a sealing member configured to form a seal between the frame and the surrounding anatomy without substantially interfering with blood flow entering the upstream portions of the frame, such as adjacent the ostia of the hepatic veins when implanted in the inferior vena cava.

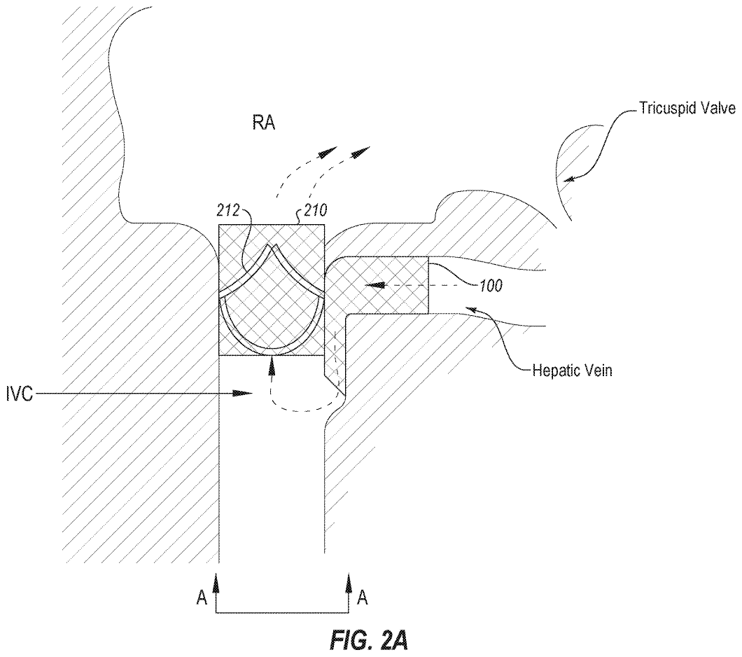

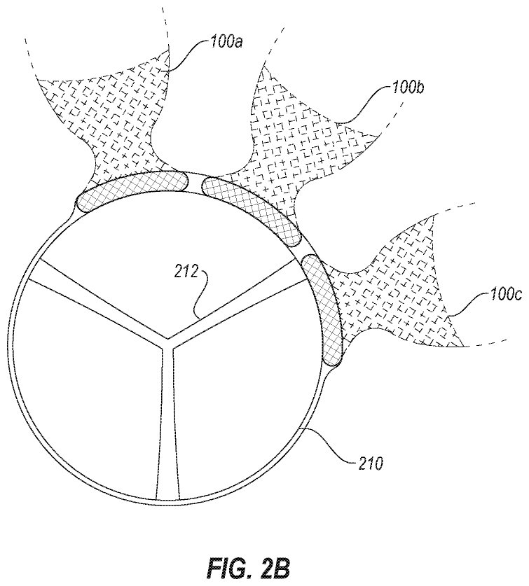

When implanting such a docking station, the docking station may occlude or close off certain vessels in the anatomy that may negatively impact the patient. For example, the sealing member of the docking station deployed within the inferior vena cava (IVC) may occlude hepatic veins. For convenience, the remainder of the examples included herein will reference the hepatic veins and the IVC, such as illustrated in FIGS. 2A-2B, although embodiments of the present disclosure may be used in conjunction with any of a variety of vessels and/or cavities. For example, if a docking station and valve were disposed further down the IVC, the renal veins might be occluded by the docking station.

To avoid the occlusion of such vessels while still enjoying the benefit of using such docking stations, embodiments of the present disclosure may include an implant configured to be deployed within one or more of the hepatic veins (e.g., the vessels which might be occluded) and provide a flow channel for blood through the hepatic veins and into the IVC (e.g., the vessel within which the docking station is deployed). For example, such an implant may include a framework of struts forming a tunnel, where the framework of struts includes an elbow separating a first span configured to be deployed into the hepatic vein, and a second span of the implant configured to extend into the IVC. The implant may also include a first opening to the tunnel associated with the first span that includes a generally circular-shaped opening within the hepatic vein. The implant may also include a second opening to the tunnel associated with the second span that may include a generally oval-shaped opening within the IVC.

In some example embodiments, docking stations/devices for prosthetic valves or THVs are illustrated as being used within the superior vena cava (SVC), inferior vena cava (IVC), or both the SVC and the IVC, although the docking stations/devices may be used in other areas of the anatomy, heart, or vasculature, such as the tricuspid valve, the pulmonary valve, the pulmonary artery, the aortic valve, the aorta, the mitral valve, or other locations. The docking stations/devices described herein may be configured to compensate for the deployed transcatheter valve or THV being smaller and/or having a different geometrical shape than the space (e.g., anatomy/heart/vasculature/etc.) in which it is to be placed. For example, the native anatomy (e.g., the IVC) may be oval, egg shaped, or another shape, while the prosthetic valve or THV may be cylindrical.

For the sake of uniformity, in the present disclosure the docking stations are typically depicted such that the right atrium end (e.g., the outflow end) is up, while the ventricular end or IVC end (e.g., the inflow end) is down unless otherwise indicated.

### First Representative Embodiment

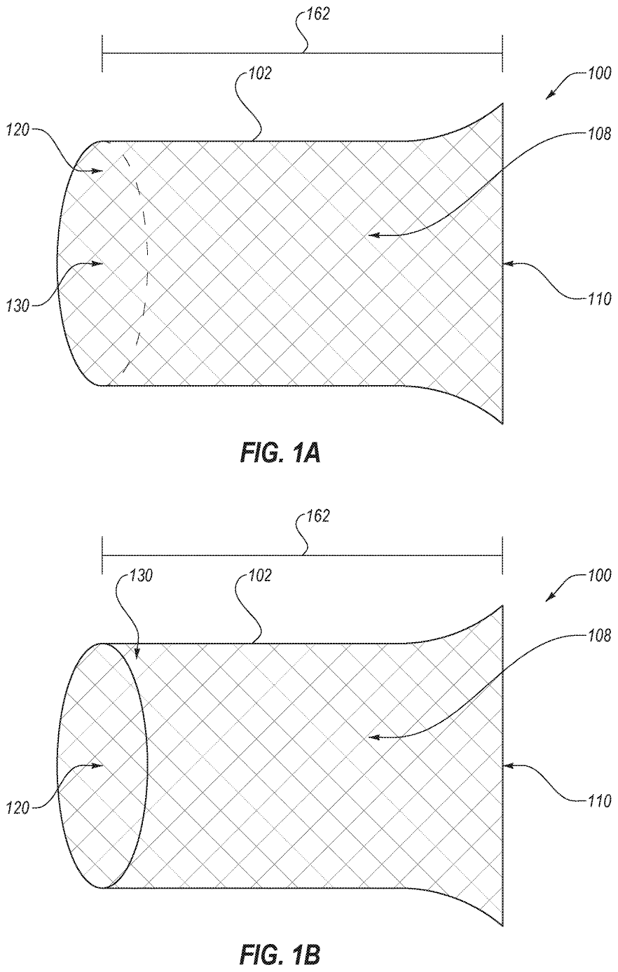

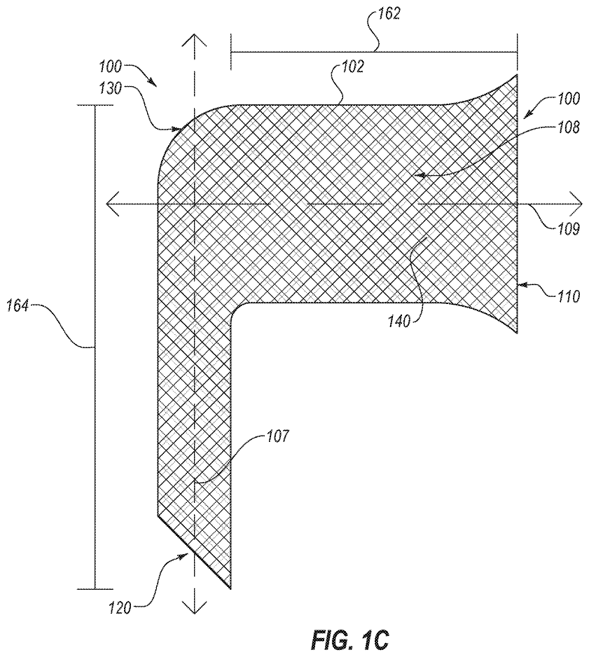

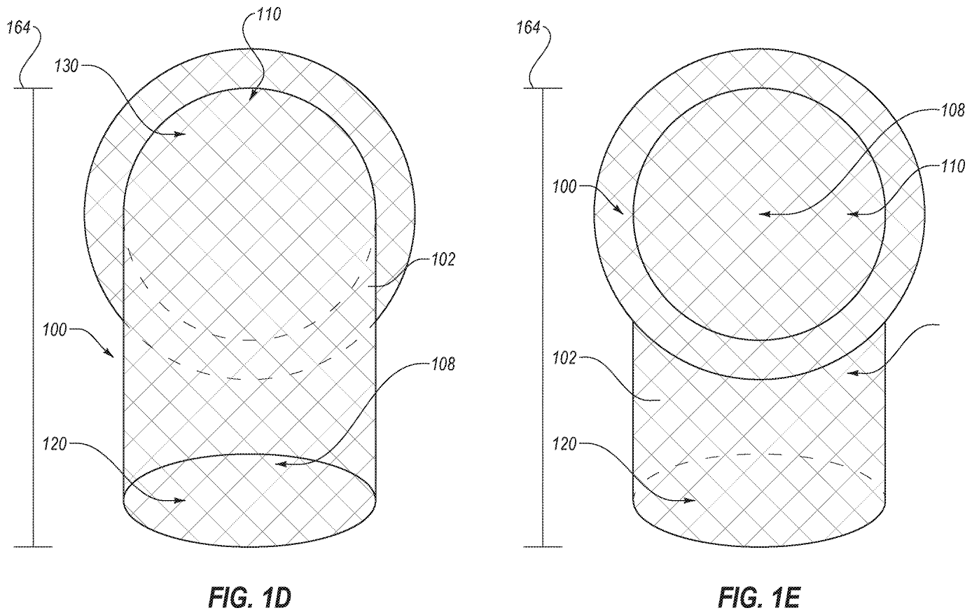

FIGS. 1A-1E illustrate various views of an implant 100 that provides a prosthetic valve support with a flow channel. FIG. 1A is a top view of the implant 100. FIG. 1B is a bottom view of the implant 100. FIG. 1C is a side view of the implant 100. FIG. 1D is a front view of the implant 100. FIG. 1E is a back view of the implant 100.

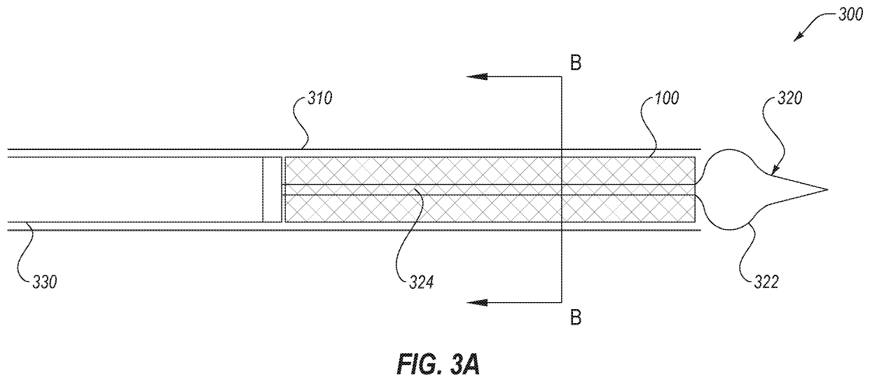

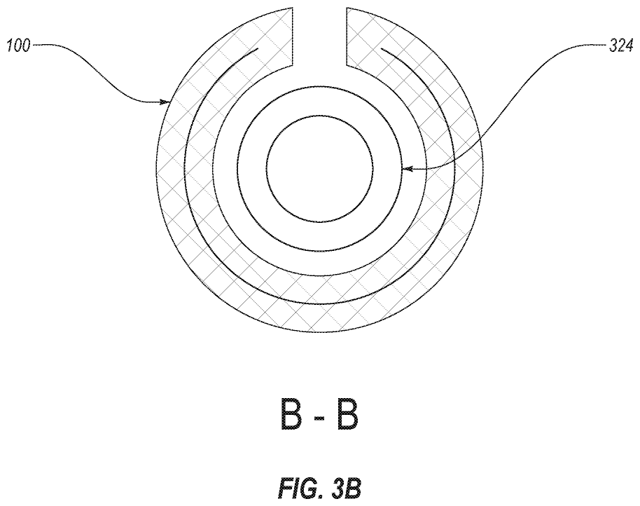

The implant 100 may include a framework 102 of struts that form a tunnel 108 that extends from a first opening 110, along a first span 162, past an elbow 130, along a second span 164, and to a second opening 120. The framework 102 may be made from a highly flexible metal, metal alloy, or polymer. Examples of metals and metal alloys that may be used include, but are not limited to, nitinol and other shape memory alloys, elgiloy, and stainless steel, but other metals and highly resilient or compliant non-metal materials may be used to make the framework 102. These materials may allow the framework 102 to be compressed to a small size, and then when the compression force is released, the framework 102 will self-expand back to its pre-compressed shape and/or the framework 102 may be expanded by inflation of a device positioned inside the framework 102. The framework 102 may also be made of other materials and be expandable and collapsible in different ways, e.g., mechanically-expandable, balloon-expandable, self-expandable, or a combination of these. For example, the implant 100 may be in a compressed state for delivery and navigation through the body of a patient to a target location (e.g., the hepatic vein) and may expand during deployment of the implant 100 to an expanded state. FIGS. 1A-1E illustrate the implant 100 in the expanded state. FIGS. 3A and 3B provide an example of the implant 100 in the compressed state.

In some embodiments, the framework 102 may be cut from a single cylinder or tube of a shape memory alloy such as nitinol, formed with the elbow 130, and then heat treated to set the shape of the framework 102. After setting the shape including the elbow 130, the implant 100 may be loaded on a delivery catheter, an example of which is illustrated in FIGS. 3A and 3B.

In some embodiments, first opening 110 may include a generally circular shaped opening to correspond to the opening of the hepatic vein. For example, when deployed the framework 102 may expand until the first opening 110 is pressed against the anatomy of the hepatic vein such that the blood flow through the hepatic vein proceeds completely through the opening 110 and into the tunnel 108. In these and other embodiments, the first span 162 may flare proximate the first opening 110 to facilitate formation of a seal between the framework 102 about the first opening 110 and the anatomy of the hepatic vein.

In some embodiments, the first span 162 may generally follow a lateral direction 109. In these and other embodiments, the first span 162 may be approximately 5 mm, 7.5 mm, 10 mm, 12.5 mm, 15 mm, 17.5 mm, 20 mm, 25 mm, 30 mm, or any range bounded by the preceding values, such as between 10 mm and 30 mm, or as another example, between 15 mm and 20 mm.

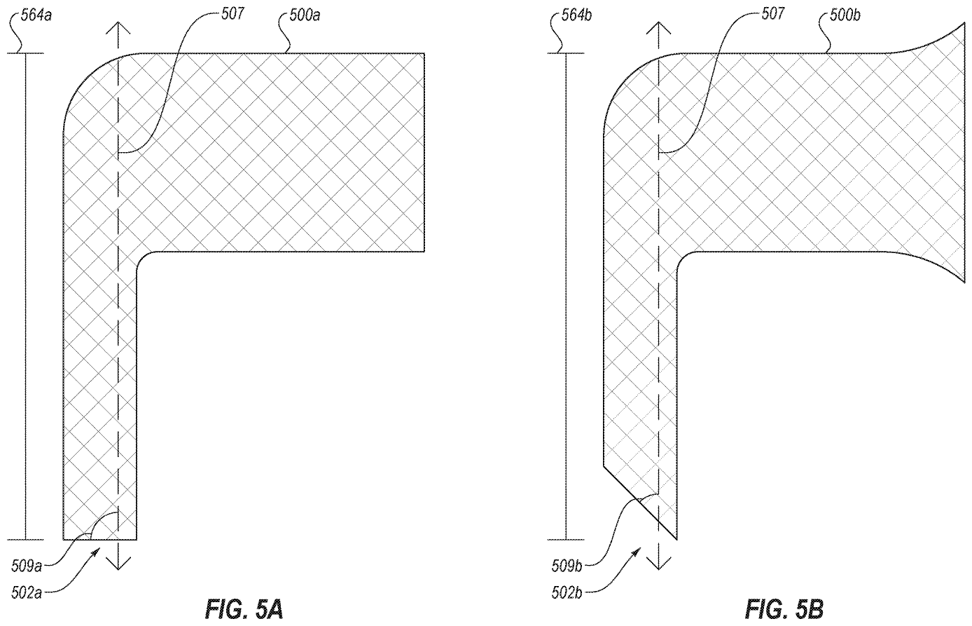

In some embodiments, the second opening 120 at the end of the second span 164 opposite the elbow 130 may include a generally oval-shaped opening. In some embodiments, the oval opening may be relatively narrow to allow for greater expansion of a docking station while still permitting the blood flow to exit the tunnel 108 via the second opening 120. In these and other embodiments, the narrowness of the oval-shaped opening may be described by reference of a minor axis of the oval shape to a major axis of the oval shape. For example, the minor axis of the oval shape may be approximately 60% of the length of the major axis, approximately 50%, approximately 40%, approximately 33% (e.g., ⅓^{rd} of the length), approximately 30%, approximately 25% (e.g., ¼^{th} of the length), approximately 20% (e.g., ⅕^{th} of the length), approximately 15% of the length, and/or approximately 10% of the length, or any ranges bounded by any of the foregoing values (such as between approximately 20% and 33% of the length). In some embodiments, the second opening 120 may be positioned to open into the IVC. Various examples of such orientation of the second opening 120 may be described with reference to FIGS. 5A and/or 5B.

In some embodiments, the second span 164 may follow a generally longitudinal direction 107. For example, the second span may span from the top of the elbow 130 to the second opening. 120. In these and other embodiments, the second span 164 may be approximately 2.5 mm, 5 mm, 7.5 mm, 10 mm, 12.5 mm, 15 mm, 17.5 mm, 20 mm, 25 mm, or any range bounded by the preceding values, such as between 5 mm and 17.5 mm.

In some embodiments, the elbow 130 may provide a junction between the first span 162 and the second span 164. The elbow 130 may provide a transition from the lateral direction 109 to the longitudinal direction 107. For example, the elbow 130 may include a bend of approximately 90°. Additionally or alternatively, the elbow 130 may include a bend of approximately 60°, 70°, 80°, 90°, 100°, 110°, 120°, or any range bounded by any of the preceding values, such as between 80° and 100°.

In some embodiments, the implant 100 may be symmetrical about a plane defined by the longitudinal direction 107 and the lateral direction 109. Additionally or alternatively, the implant 100 may be asymmetrical about the plane defined by the longitudinal direction 107 and the lateral direction 109. In some embodiments, the implant 100 may be symmetrical about a plane defined by the longitudinal direction 107 and the lateral direction 109 when in an expanded state outside of a patient, but may expand in an asymmetric way such that when deployed into an expanded state within a patient, the implant 100 may not be symmetric about the plane defined by the longitudinal direction 107 and the lateral direction 109.

In some embodiments, such as that illustrated in FIG. 1C, the implant 100 may include a sealing material 140 disposed on the outside of the tunnel 108 which may act as a sealing skirt for the implant 100. The sealing material 140 may guide the blood flow through the tunnel 108 without having the blood leak out of the tunnel 108 before reaching the second opening 120. For example, the sealing material 140 may provide a seal between the framework 102 proximate the first opening 110 and an interior surface of the hepatic vein. That is, the sealing material 140 and the framework 102 prevent or inhibit blood from flowing around the implant 100 without flowing into the tunnel 108. In some embodiments, the sealing material 140 may be disposed on the outside of the framework 102 to soften an interface between the framework 102 and the anatomy of the patient. Additionally or alternatively, the sealing material 140 may be disposed on an inner surface of the tunnel 108 formed by the framework 102. In some embodiments, the sealing material 140 may be a fabric material, polymer material, or other material.

FIGS. 2A and 2B illustrate the implant 100 of FIGS. 1A-1E deployed within a patient. FIG. 2A is a cutaway view of anatomy of the patient proximate the IVC and the right atrium RA with a deployed docking station 210 and valve 212, and with the implant 100 also deployed. FIG. 2B is a view of the anatomy of FIG. 2A along the view line A-A illustrated in FIG. 2A.

As illustrated in FIG. 2A, the docking station 210 may be deployed proximate a juncture between the RA and the IVC. The docking station 210 may include a valve framework within which the valve 212 may be deployed. For example, the valve 212 may be deployed within the valve framework of the docking station 210 such that blood flow (designated by the dashed arrows) may flow from the IVC into the RA but will not flow back from the RA into the IVC.

Along a side of the docking station 210 (e.g., abutting the docking station 210), the implant 100 may be deployed within the hepatic vein such that the implant 100 provides a flow path for the blood flow from within the hepatic vein, through the implant 100 past the docking station 210, and out into the IVC. The blood may then flow from the IVC into the RA via the valve 212.

In some embodiments, the docking station 210 may create a seal against the anatomy of the patient such that blood cannot flow or may only flow in a very limited manner (e.g., a small mount of seepage) from the RA back into the IVC around the peripheral edge of the docking station 210. For example, the docking station 210 may include a sealing skirt that extends from the valve framework to an edge of the docking station 210 such that the sealing skirt of the docking station 210 may create a seal about the junction of the IVC and the RA.

As illustrated in FIG. 2B, one or more of the implants 100 (such as the first, second and third implants 100a, 100b, and/or 100c, respectively) may be used to provide a tunnel for blood flow in each of the hepatic veins.

In operation, a guide catheter may be guided through the patient to the hepatic veins, and the first implant 100a, within a delivery catheter, may be guided through the patient to be within a first hepatic vein. After being disposed within the hepatic vein, the delivery catheter may deploy the first implant 100a such that part or all of a first span 162 is disposed within the hepatic vein (as illustrated in FIG. 2B). The second and third implants 100b and 100c may likewise be guided through the patient to the second and third hepatic veins and deployed therein in a similar or comparable manner.

After deploying the implants 100, the docking station 210 may be guided to the target location for the valve 212, such as proximate the juncture between the IVC and the RA. As the docking station 210 is expanded, the docking station 210 may form a seal against the anatomy of the patient.

When expanding the docking station 210, the rigidity of the implant 100 may be greater in the second span 164 than in the anatomy of the IVC and/or the hepatic vein. For example, as the docking station 210 expands, it may press against the implant 100 forcing the anatomy of the IVC outwards slightly rather than collapsing the implant 100. In these and other embodiments, by retaining the oval-shaped second opening 120 even when the docking station 210 is pressing against the implant 100, the flow of blood out of the hepatic veins and into the IVC may be maintained, even if the sealing elements of the docking station 210 overlap with the hepatic veins.

In some embodiments, the sealing skirt of the implant 100 may guide the blood flow through the implant 100 and into the IVC while the sealing skirt of the docking station 210 prevents the blood flow from flowing back from the RA into the IVC. Stated another way, the sealing material 140 of the implant 100 and the sealing skirt of the docking station 210 may prevent blood flow between the IVC and the RA except through the valve 212, while permitting blood flow from the hepatic vein through the first span 162 of the implant 100, the second span 164 of the implant 100, and into the IVC.

FIGS. 3A and 3B illustrates the implant 100 of FIGS. 1A-1E loaded on a delivery catheter 300. FIG. 3A illustrates a cut-away side view of the implant 100 loaded on the delivery catheter 300. FIG. 3B is a view of the implant loaded on the delivery catheter 300 along the view line B-B illustrated in FIG. 3A.

The delivery catheter 300 may include an outer sheath 310 and a main body 330. The delivery catheter 300 may also include a guiding tip 320, a flare 322 proximate the guiding tip 320, and a loading shaft 324. For example, the main body 330 may include a flexible and guidable shaft used to move the delivery catheter 300 through the body of the patient. The guiding tip 320 may include a tapered end that flares out at the flare 322. The flare 322 may extend as far as or farther than the outer sheath 310 such that the outer sheath 310 does not snag or catch on anatomy of the patient as the delivery catheter 300 navigates through the patient.

In some embodiments, the loading shaft 324 may include an extension of the main body 330 or may include a more narrow portion coupled to the main body 330. In these and other embodiments, the loading shaft 324 may include a portion of the delivery catheter 300 with a reduced diameter such that the implant 100 in the compressed state may be loaded on the loading shaft 324 and still include a total outer diameter (e.g., the outer diameter of the combination of the loading shaft 324 and the implant 100) that will fit within the outer sheath 310.

As illustrated in FIG. 3A, the implant 100 may be compressed into the compressed state. The implant 100 may be compressed into the compressed state in any of a variety of manners or using any of a variety of approaches. For example, the implant 100 may be radially compressed in a generally uniform manner about the loading shaft 324. In some embodiments, this may occur as the implant 100 is loaded on the loading shaft 324. For example, the implant 100 may be placed around the loading shaft 324 in an uncompressed state and then as the outer sheath 310 is slid up and over the implant 100, the implant 100 may be compressed into the compressed state about the loading shaft 324.

As illustrated in FIG. 3B, in some embodiments the implant 100 may be compressed in a two-step approach in which the implant 100 is first flattened forming a two-layered component. The flattened implant 100 is then bent back around itself about the loading shaft 324 such that there are two layers of the framework 102 of the implant about a majority of the loading shaft 324 rather than only a single layer of the framework 102 about the loading shaft 324 when the implant 100 is compressed radially.

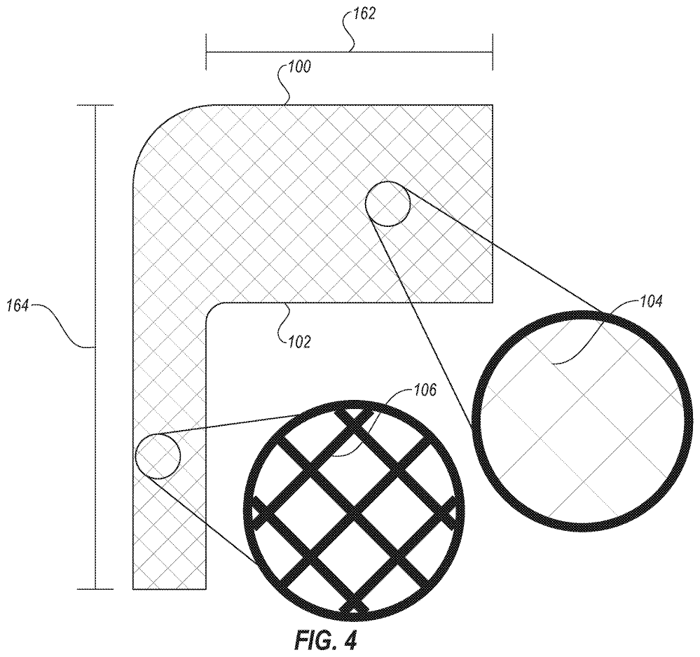

FIG. 4 is a side view of the implant of FIG. 1A-1E with a close-up view of struts with different thicknesses in the same device, such as first struts 104 in a general region of the first span 162 and second struts 106 in a general region of the second span 164.

As illustrated in FIG. 4, the first struts 104 may include a lighter, thinner, more flexible framework relative to the second struts 106 that may permit greater flexibility and adaptability for the first span 162. In this manner, the first struts 104 may permit the first span 162 to expand within the hepatic vein to create a seal against the wall of the hepatic vein while decreasing the likelihood of damaging the native tissue. Stated another way, the thinner first struts 104 may prevent the implant 100 in the first span 162 from pushing outwards on the walls of the hepatic veins with too much force, instead being more pliable to more closely align with the natural anatomy of the hepatic veins. In these and other embodiments, the first span 162 may push against the walls of the hepatic vein and may expand beyond the native shape of the hepatic vein. However, the thinner first struts 162 may decrease the likelihood of damage as the expansion occurs and/or may reduce the degree to which the expansion occurs.

As illustrated in FIG. 4, the second struts 106 may include a heavier framework relative to the first struts 104. The heavier second struts 106 may facilitate the second span 164 retaining its shape and rigidity. For example, when an associated docking station is expanded against the implant 100 and presses against the second span 164, the heavier second struts 106 may facilitate the second span 164 maintaining the oval shape. In some embodiments, the entire second span 164 includes the heavier second struts 106. Additionally or alternatively, only portions of the second span 164 may include the heavier second struts 106, such as the curved portions about the minor axis of the oval shape of the second opening 120. In some embodiments, the entire elbow 130 or portions thereof may also include the heavier second struts 106.

In some embodiments, the difference between the first struts 104 and the second struts 106 may be realized when the framework 102 is cut out of a single tube of material. For example, the cutting may remove additional material from around the first struts 104 while leaving more material when cutting the second struts 106. In some embodiments, both the first struts 104 and the second struts 106 may be cut to a same or similar size and a reinforcing material may be added to the second struts 106 after cutting out the struts to create the heavier second struts 106.

In some embodiments, a thickness of the first struts 104 may be expressed in dimension relative to the second struts 106. In some embodiments, a given strut of the first struts 104 may include a thickness or diameter that is approximately 60% of the second struts, 50% (e.g., ½) of the second struts, 40% of the second struts, 33% (e.g., ⅓) of the second struts, 30% of the second struts, 25% (e.g., ¼) of the second struts, 20% (e.g., ⅕) of the second struts, 10% of the second struts, or any range bounded by any of the preceding values, such as a given strut of the first struts 104 including a thickness or diameter that is between approximately 60% and approximately 20% of the thickness of the second struts 106. In some embodiments, the thickness of the first struts 104 and/or the second struts 106 may be described by raw measurements, such as a thickness of approximately 0.25 mm, 0.5 mm. 0.75 mm, 1 mm, 1.25 mm, 1.5 mm, 2 mm, 2.5 mm, 3 mm, 3.5 mm, 4 mm, 5 mm, or any other range bounded by the preceding values. For example, the first struts 104 may include a thickness between 0.25 and 1.5 mm, and the second struts 106 may include a thickness between 1 mm and 3.5 mm.

FIGS. 5A and 5B illustrate side views of implants 500a/500b, respectively, with different second openings 502a/502b, respectively, to create the flow channel. The implants 500a/500b may make an angle 509 (such as the angles 509a/509b respectively) with a longitudinal direction 507.

The implants 500a/500b may be similar or comparable to the implant 100. FIGS. 5A and 5B may illustrate that the implants 500a/500b may include different orientations and/or shapes of the second openings, two examples of which are illustrated in FIGS. 5A and 5B.

As illustrated in FIG. 5A, the second opening 520a may make the angle 509a with the longitudinal direction 507 running generally parallel with a second span 564a of the implant 500a. For example, as illustrated in FIG. 5A, the angle 509a may include approximately 90°, e.g., be normal to the longitudinal direction 507.

As illustrated in FIG. 5B, the second opening 520b may make the angle 509b with the longitudinal direction 507 running generally parallel with a second span 564b of the implant 500b. For example, as illustrated in FIG. 5B, the angle 509b may include approximately 45°.

While illustrated as 90° and 45° in FIGS. 5A and 5B respectively, it will be appreciated that any of a variety of angles are contemplated within the scope of the present disclosure. For example, the angle 509 may include 10°, 20°, 30°, 40°, 45°, 50°, 60°, 70°, 80°, 90°, 100°, 110°, 120°, and/or any range of angles bounded by the foregoing, such as between 30° and 100°.

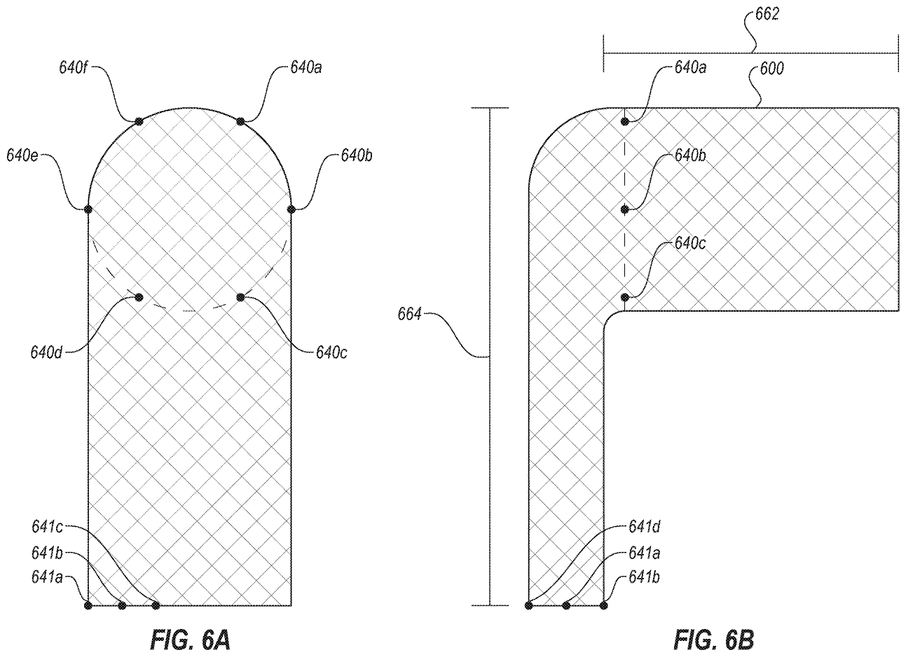

FIGS. 6A and 6B illustrate an example implant 600 that includes radiopaque markers 640 (such as the radiopaque markers 640a-640f) and/or 641 (such as the radiopaque markers 641a-641d) to facilitate placement and/or orientation of the example implant 600. The implant 600 may be similar or comparable to the implant 100 and/or the implants 500a/500b. For example, the implant 600 may include a first span 662 and a second span 664 that may be similar or comparable to the first span 162 and the second span 164.

In some embodiments, a series of radiopaque markers 640 about a circumference of the implant 600 may be included to facilitate guidance, orientation, and/or placement of the implant 600 within a patient. For example, the radiopaque markers 640 may facilitate guidance, orientation, and otherwise facilitate a procedure of guiding the implant 600 through the body of the patient to the target location (e.g., the hepatic vein), deploying the implant 600, and/or otherwise positioning the implant 600 in the desired position. For example, the radiopaque markers 640 may facilitate a view into an axial or radial orientation, state of deployment, or other information for the clinician when performing a procedure involving the implant 600. The radiopaque markers 640 may be made of any radiopaque material, such as tantalum, bismuth, iodine, barium, or gold.

In some embodiments, the radiopaque markers 640 may be positioned between the first opening 610 and the elbow 630. For example, the radiopaque markers 640 may be positioned at an end of the first span 662 and/or at the start of the elbow 630 proximate the first span 662. Stated another way, the radiopaque markers 640 may be located closer to the elbow 630 than to the first opening 610. By providing the radiopaque markers 640 at or towards the end of the first span 662, the clinician may align the radiopaque markers 640 with an edge of the hepatic vein leading into the IVC to verify that the implant 600 is in the target position such that when deployed, the first span 662 will be within the hepatic vein and the second span 664 will be within the IVC.

In some embodiments, the implant 600 may include the radiopaque markers 641 to facilitate the orientation of the implant 600. For example, the radiopaque markers 641 may facilitate positioning the implant 600 such that the second span 664 extends from the hepatic vein down and away from the RA and along the IVC rather than extending from the hepatic vein up the IVC towards the RA. For example, the radiopaque markers 641 may be positioned only partway around or unevenly around the circumference of the implant 600 such that when observed, a clinician may be able to rotate the implant 600 before deployment such that when the implant 600 expands to its expanded form, the second span 664 extends along the IVC instead of extending up towards the RA.

In some embodiments, the implant 600 may include the radiopaque markers 640 being distributed only partway around the circumference of the implant 600 and/or being unevenly spaced about the circumference of the implant 600. By providing a visually distinguishable distribution of the radiopaque markers 640, a clinician may utilize the distinction to orient the implant 600. In some embodiments, a single radiopaque marker 640 may be used, such as on the very top of the implant 600 at the end of the first span 662 and/or on the very bottom of the implant 600 at the end of the first span 662. In these and other embodiments, the radiopaque markers 640 may be usable to both identify how far within the hepatic vein the implant 600 is to be guided as well as facilitating orienting the implant 600 in the appropriate direction prior to or during deployment of the implant 600.

While examples of locations, distributions, and numbers of radiopaque markers 640 and/or 641 are illustrated in FIGS. 6A and 6B, it will be appreciated that any number and/or location of radiopaque markers are contemplated within the scope of the present disclosure.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implant comprising: a framework of struts forming a tunnel, the framework of struts including: an elbow separating a first span and a second span of the implant; a first opening to the tunnel associated with the first span including a generally circular-shaped opening; and a second opening to the tunnel associated with the second span including a generally oval-shaped opening.

Example 2. Any of the foregoing examples, such as example 1, further comprising a sealing skirt enclosing the tunnel.

Example 3. Any of the foregoing examples, such as example 2, wherein the sealing skirt covers an entire outer surface of the framework of struts.

Example 4. Any of the foregoing examples, such as any of examples 1-3, wherein the framework of struts from the second opening to the elbow includes heavy struts.

Example 5. Any of the foregoing examples, such as any of examples 1-4, wherein the framework of struts from the first opening to the elbow includes flexible struts.

Example 6. Any of the foregoing examples, such as any of examples 1-5, further comprising radiopaque markers around a circumference of the tunnel.

Example 7. Any of the foregoing examples, such as example 6, wherein the radiopaque markers are disposed between the elbow and the first opening.

Example 8. Any of the foregoing examples, such as example 7, wherein the radiopaque markers are closer to the elbow than to the first opening.

Example 9. Any of the foregoing examples, such as any of examples 1-8, wherein the first span follows a lateral direction and the second span follows a longitudinal direction.

Example 10. Any of the foregoing examples, such as example 9, wherein the second opening is approximately normal to the longitudinal direction.

Example 11. Any of the foregoing examples, such as example 9, wherein the second opening is at an angle of between 30° and 90° as measured from the longitudinal direction.

Example 12. Any of the foregoing examples, such as example 9-11, wherein the implant is generally symmetrical about a plane defined by the lateral direction and the longitudinal direction.

Example 13. Example 14. Any of the foregoing examples, such as example 13, wherein the first span is between 15 and 20 mm.

Any of the foregoing examples, such as any of examples 1-12, wherein the first span is between 10 and 30 mm.

Example 15. Any of the foregoing examples, such as any of examples 1-14, wherein the framework of struts is expandable between a compressed state and an expanded state.

Example 16. Any of the foregoing examples, such as example 15, wherein the framework is made of a self-expanding material.

Example 17. Any of the foregoing examples, such as example 16, wherein the framework is made of nitinol.

Example 18. Any of the foregoing examples, such as any of examples 15-17, wherein the compressed state includes a flattened and rolled configuration.

Example 19. Any of the foregoing examples, such as example 15-17, wherein the compressed state includes a radially compressed state.

Example 20. Any of the foregoing examples, such as any of examples 1-19, wherein the elbow includes a bend of approximately 90°.

Example 21. A method of deploying an artificial valve, comprising: guiding a delivery catheter through a body of a patient to a first hepatic vein; deploying a first implant so that a first span of the first implant is disposed within the first hepatic vein and a second span of the first implant is disposed outside of the first hepatic vein, a first elbow separating the first span and the second span of the first implant; and deploying a valve framework at a junction between an inferior vena cava (IVC) and a right atrium (RA) of the patient, the valve framework abutting at least part of the second span of the first implant.

Example 22. Any of the foregoing examples, such as example 21, further comprising, prior to deploying the valve framework: guiding the delivery catheter to a second hepatic vein; and deploying a second implant so that a first span of the second implant is disposed within the second hepatic vein and a second span of the second implant is disposed outside of the second hepatic vein, a second elbow separating the first span and the second span of the second implant, wherein the valve framework further abuts at least part of the second span of the second implant.

Example 23. Any of the foregoing examples, such as any of examples 21-22, wherein the first implant includes radiopaque markers about a circumference of the first span of the first implant, and wherein deploying the first implant includes orienting the radiopaque markers at an entrance to the first hepatic vein.

Example 24. Any of the foregoing examples, such as any of examples 21-23, further comprising: delivering an artificial valve to the valve framework; and deploying the artificial valve into the valve framework.

Example 25. Any of the foregoing examples, such as example 24, wherein the first implant includes a first sealing skirt and the valve framework includes a second sealing skirt.

Example 26. Any of the foregoing examples, such as example 25, wherein the first sealing skirt and the second sealing skirt prevent blood flow between the inferior vena cava and the right atrium except through the artificial valve, while permitting blood flow from the hepatic vein through the first span of the first implant, the second span of the first implant, and into the inferior vena cava.

Example 27. Any of the foregoing examples, such as example 26, wherein the first implant includes a tunnel enclosed by the first sealing skirt through which the blood flows from the hepatic vein to the inferior vena cava.

Example 28. Any of the foregoing examples, such as any of examples 21-27, wherein the first implant includes more rigid material in the second span of the first implant than in the first span of the first implant such that the second span of the first implant does not collapse when the valve framework abuts the at least part of the second span of the first implant.

Example 29. Any of the foregoing examples, such as example 28, wherein deploying the valve framework includes displacing a wall of the inferior vena cava by the second span of the first implant because of the valve framework pressing against the first implant.

Example 30. An implant comprising: a framework of struts that forms a tunnel, the framework of struts including: a first span, a second span, and an elbow between the first span and the second span; a first opening to the tunnel associated with the first span including a generally circular-shaped opening; and a second opening to the tunnel associated with the second span including a generally oval-shaped opening.

Example 31. A method of deploying an artificial valve, comprising: guiding a delivery catheter through a body of a patient to a first hepatic vein; deploying a first implant so that a first span of the first implant is disposed within the first hepatic vein and a second span of the first implant is disposed outside of the first hepatic vein, a first elbow joining the first span and the second span of the first implant; and deploying a valve framework at a junction between an inferior vena cava (IVC) and a right atrium (RA) of the patient, the valve framework abutting at least part of the second span of the first implant.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope and spirit of these claims.

What is claimed is:

1. An implant comprising:
a framework of struts forming a tunnel, the framework of struts including:
an elbow separating a first span and a second span of the implant;
a first opening to the tunnel associated with the first span including a generally circular-shaped opening; and
a second opening to the tunnel associated with the second span including a generally oval-shaped opening.

2. The implant of claim 1, further comprising a sealing skirt enclosing the tunnel.

3. The implant of claim 2, wherein the sealing skirt covers an entire outer surface of the framework of struts.

4. The implant of claim 1, wherein the framework of struts from the second opening to the elbow includes heavy struts.

5. The implant of claim 1, wherein the framework of struts from the first opening to the elbow includes flexible struts.

6. The implant of claim 1, further comprising radiopaque markers around a circumference of the tunnel.

7. The implant of claim 6, wherein the radiopaque markers are disposed between the elbow and the first opening.

8. The implant of claim 7, wherein the radiopaque markers are closer to the elbow than to the first opening.

9. The implant of claim 1, wherein the first span follows a lateral direction and the second span follows a longitudinal direction.

10. The implant of claim 9, wherein the second opening is approximately normal to the longitudinal direction.

11. The implant of claim 9, wherein the second opening is at an angle of between 30° and 90° as measured from the longitudinal direction.

12. The implant of claim 9, wherein the implant is generally symmetrical about a plane defined by the lateral direction and the longitudinal direction.

13. The implant of claim 1, wherein the first span is between 10 and 30 mm.

14. The implant of claim 13, wherein the first span is between 15 and 20 mm.

15. The implant of claim 1, wherein the framework of struts is expandable between a compressed state and an expanded state.

16. The implant of claim 15, wherein the compressed state includes a flattened and rolled configuration.

17. The implant of claim 15, wherein the compressed state includes a radially compressed state.

18. The implant of claim 1, wherein the elbow includes a bend of approximately 90°.

19. A method of deploying an artificial valve, comprising:
guiding a delivery catheter through a body of a patient to a first hepatic vein;
deploying a first implant so that a first span of the first implant is disposed within the first hepatic vein and a second span of the first implant is disposed outside of the first hepatic vein, a first elbow separating the first span and the second span of the first implant; and
deploying a valve framework at a junction between an inferior vena cava (IVC) and a right atrium (RA) of the patient, the valve framework abutting at least part of the second span of the first implant.

20. The method of claim 19, further comprising, prior to deploying the valve framework:
guiding the delivery catheter to a second hepatic vein; and
deploying a second implant so that a first span of the second implant is disposed within the second hepatic vein and a second span of the second implant is disposed outside of the second hepatic vein, a second elbow separating the first span and the second span of the second implant,
wherein the valve framework further abuts at least part of the second span of the second implant.
